(19) United States
(12) Patent Application Publication
(10) Pub. No.: US 2026/0124264 A1
(43) Pub. Date: May 7, 2026

(54) ANTI-INFLAMMATORY COMPOSITION

(71) Applicant: FermentationExperts A/S, Baekke (DK)

(72) Inventor: Jens Hoffner Legarth, Baekke (DK)

(73) Assignee: FermentationExperts A/S, Baekke (DK)

(21) Appl. No.: 19/379,287

(22) Filed: Nov. 4, 2025

Related U.S. Application Data
(62) Division of application No. 17/601,395.

(30) Foreign Application Priority Data
Apr. 6, 2019 (DK) PA 2019 00425

(51) Int. Cl. A61K 36/31; A61K 36/03; A61P 29/00

(52) U.S. Cl. CPC A61K 36/31; A61K 36/03; A61P 29/00; A61K 2236/19

(57) ABSTRACT

A composition having a non-polar fraction obtained from at least one fermented plant material is provided. The at least one plant material has been subjected to a lactic acid fermentation.

### TECHNICAL FIELD OF THE INVENTION

The present invention relates to a composition, obtained from a fermented plant material, having anti-inflammatory activity and capable of suppressing interleukin-12, interleukin-1β, and/or interleukin-6 activity; and/or suppressing of tumor necrosis factor alpha (TNF-alpha or TNF-α) activity; and/or upregulating interleukin-10, interleukin-1Rα, interleukin-4, interleukin-11, interleukin-13; or transforming growth factor beta (TGF-β); by administration of the composition to a mammal, which composition may be used in the preparation of an anti-inflammatory substance for the treatment, alleviation or prophylaxis of a variety of disorders, including the treatment of pathological conditions associated with inflammation.

### BACKGROUND OF THE INVENTION

Herbal medicines have always been used to fight diseases and infections in humans and animals, and plants has shown to comprise many constituents having positive nutritional and pharmacological effects in the human and animal organism. Despite the long-term knowledge and search for providing products and isolated active ingredients from plants, researchers seldom succeed because of the complexity and interplay between the individual components of the plants. Examples of potent extracts from plants that are well integrated in modern medical treatment can be found in drugs such as Morphine for treatment of severe pain, Digitalis for treatment of heart disease, Taxole for cancer treatment.

Furthermore, reaction products from plants that have been chemically or microbial treated have shown to be very interesting. However, predicting the interesting reaction products and how to provide the relevant reaction product is very difficult or even impossible.

Often it is also difficult to predict that one reaction product may be responsible for the activity of e.g. a fermented plant material, as the fermented plant material is a complex mixture of an enormous number of different compounds where some products have pharmacological activity by itself and other only possess the pharmacological activity when in the complex mixture.

Mammals, like humans and animals, subjected to an inflammation express an increased activity in one or more cytokines. Two of the most common cytokins that are followed when dealing with an inflammation may be selected from the group consisting of interleukin-12, interleukin-1β, interleukin-6, tumor necrosis factor alpha (TNF-alpha or TNF-α), interleukin-10, interleukin-1Ra, interleukin-4, interleukin-11, interleukin-13; and transforming growth factor beta (TGF-β). In particular, interleukin 10, interleukin-12 and tumor necrosis factor alpha (TNF-alpha or TNF-α) may be followed.

Cytokines are small, secreted polypeptides from higher eukaryotes which are responsible for intercellular signal transduction and which affect the growth, division and functions of other cells. They are potent, pleiotropic polypeptides that, e.g. via corresponding receptors, act as local or systemic intercellular regulatory factors, and therefore play crucial roles in many biologic processes, such as immunity, inflammation, and hematopoiesis. Cytokines are produced by diverse cell types including fibroblasts, endothelial cells, epithelial cells, macrophages/monocytes, and lymphocytes.

Current anti-inflammatory drugs are designed to act in an indirect manner, by blocking the action of e.g. interleukin-12, interleukin-1β, and/or interleukin-6 activity; and/or suppression of tumor necrosis factor alpha (TNF-alpha or TNF-α) activityby binding to it and hereby prevents it from signaling the receptors for TNF-α on the surface of cells. This type of blocking has some serious side effects, of which some is infections such as tuberculosis, sepsis and fungal infections and possible increased cancer incidence, and an increased cytokine activity is still provided.

Hence, there is a need in the industry for new products and compounds for the efficient treatment, alleviation and/or prophylaxis of an inflammatory disease or disorder in a mammal.

### SUMMARY OF THE INVENTION

Thus, an object of the present invention relates to a composition obtained from a fermented plant material, having anti-inflammatory activity and capable of:
suppressing interleukin-12, interleukin-1β, and/or interleukin-6 activity; and/or
suppressing of tumor necrosis factor alpha (TNF-alpha or TNF-α) activity; and/or
upregulating interleukin-10, interleukin-1Ra, interleukin-4, interleukin-11, interleukin-13; or transforming growth factor beta (TGF-β).

In particular, it is an object of the present invention to provide a new product and a new compound for the efficient treatment, alleviation and/or prophylaxis of an inflammatory disease or disorder in a mammal, and which product or compound solves the above-mentioned problems of the prior art with activity and side effects.

Thus, one aspect of the invention relates to a composition comprising a non-polar fraction obtained from at least one fermented plant material.

Another aspect of the present invention relates to an anti-inflammatory substance comprising the composition according to the present invention, for use in the treatment, alleviation and/or prophylaxis of an inflammatory disease or disorder in a mammal.

### BRIEF DESCRIPTION OF THE FIGURES

FIG. 1 shows a LC-QTOF chromatogram illustrating a metabolite profiling of the fermented plant material and illustrates two significant peak one being most significant. The one peak (a) illustrates the non-polar fraction or the fatty acid fraction, the modified fatty acid fraction according to the present invention. The other marked peak, peak (b) illustrates HDMPPA, 3-(4′-hydroxyl-3′,5′-dimethoxyphenyl)propionic acid (a kimchi compound).

FIG. 2 shows the effect of the composition and/or the anti-inflammatory substance according to the present invention on inflammation induced by NCFM MOI. The figure shows that the composition and/or the anti-inflammatory substance according to the present invention has a strong suppression on the IL-12 activity and expression see column (m) and column (n) relative to column (d) which is the control showing the effect of the untreated infected sample, column (d). Column (m) and column (n) also demonstrates that a dose response effect in the suppression of IL-12 is to be found with the composition and/or the anti-inflammatory substance according to the present invention. Column (m) has twice the amount of composition and/or the anti-inflammatory substance according to the present invention than the amount found in the test illustrated in column (n),

FIG. 3 shows the effect of the composition and/or the anti-inflammatory substance according to the present invention on inflammation induced by NCFM MOI. The figure shows that the composition and/or the anti-inflammatory substance according to the present invention has a strong suppression on the TNF-alpha activity and expression see column (m) relative to column (d) which is the control showing the effect of the untreated infected sample, column (d),

FIG. 4 shows the effect of the composition and/or the anti-inflammatory substance according to the present invention on inflammation induced by LPS (Lipopolysaccharides). The figure shows that the composition and/or the anti-inflammatory substance according to the present invention has a strong suppression on the IL-12 activity and expression see column (h) relative to column (c) which is the control showing the effect of the untreated infected sample, column (c), and

FIG. 5 shows the effect of the composition and/or the anti-inflammatory substance according to the present invention on inflammation induced by LPS (Lipopolysaccharides). The figure shows that the composition and/or the anti-inflammatory substance according to the present invention has a strong suppression on the TNF-alpha activity and expression see column (h) relative to column (c) which is the control showing the effect of the untreated infected sample, column (c).

The present invention will now be described in more detail in the following.

### DETAILED DESCRIPTION OF THE INVENTION

Anti-inflammatory substances may in general have two ways of acting. Either it may be designed to act in an indirect manner, by blocking the action of e.g. interleukin-12 (IL-12) and/or TNF-alpha by binding to it and hereby prevents it from signaling the receptors for TNF-alpha or the IL-12 on the surface of cells; or the anti-inflammatory substances may act in a direct manner suppressing the activity and/or the expression of IL-12 and/or TNF-alpha

In the present context, the terms “suppressor” or “suppressing” relates to the broad definition, and is to be given its ordinary and customary meaning to a person of ordinary skill in the art (and is not to be limited to a special or customized meaning), and refers without limitation to a molecule (e.g., natural or synthetic compound) that can decrease at least one activity of TNF-alpha or IL-12. In other words, in the present context the term “suppressor” to the change in the activity of TNF-alpha or IL-12, if there is a statistically significant change in the amount of TNF-alpha or IL-12 measured, in TNF-alpha or IL-12 activity, or in TNF-alpha or IL-12 detected extracellularly and/or intracellular in an assay performed with a suppressor, compared to the assay performed without the suppressor.

In general, TNF-alpha suppressors reduce the physiological function of TNF-alpha or IL-12, for example by reducing secretion of TNF-alpha or IL-12, and thus are useful in the treatment of diseases where TNF-alpha or IL-12 may be pathogenic, directly or indirectly.

Hence, in a preferred embodiment of the present invention relates to a composition comprising a non-polar fraction obtained from at least one fermented plant material.

In a preferred embodiment of the present invention the non-polar fraction may be a fraction comprising one or more fatty acid compounds. Preferably, the non-polar fraction may comprise one or more modified fatty acid compounds.

The non-polar fraction may comprise several different structures of fatty acid compounds, preferably, modified fatty acid compounds.

In an embodiment of the present invention the non-polar fraction may comprise one or more fatty acid compounds. Preferably, the one or more fatty acid compounds may be at least one C18-fatty acid compound or at least one modified C18-fatty acid compound.

In a further embodiment of the present invention the at least one fatty acid compound and/or the at least one C18 compound is at least one linolenic acid compound, a modified linolenic acid compound or a derivative thereof; preferably, the at least one C18 compound is at least one modified linolenic acid compound or a derivative thereof.

In an embodiment of the present invention the one or more fatty acid compounds or the one or more modified fatty acid compounds may be characterised by a significant base peak intensity at a retention time from 21-25 minutes when determined in a LC-QTOF analysis, such as a significant base peak intensity at a retention time from 21.5-24 minutes, e.g. a significant base peak intensity at a retention time from 22-23 minutes.

In the present invention the term “fatty acid compound” relates to a carboxylic acid with a long aliphatic chain, which is either saturated or unsaturated. The term “modified fatty acid compound” relates to a fatty acid compound which has been changed in the structure relative to the fatty acid compound originally present in the at least one plant material that the modified fatty acid compound is derived from.

In an embodiment of the present invention the composition may be an isolated composition separated from the at least one fermented plant material. Hence, the composition may constitute a fraction of the fermented plant material.

In the present context, the term “fraction” relates to specific parts separated from the one or more fermented plant material. Unlimited examples of specific fractions may include isolated proteins, isolated enzymes, isolated lipid fractions, isolated fatty acid compounds, isolated fibrous fractions, combinations of fractions etc.

In an embodiment of the present invention the non-polar fraction may be isolated from at least one fermented plant material. Preferably. The non-polar fraction according to the present invention does not comprises at least one of a fibrous material; a protein material; an enzyme material or microorganisms from the at least one fermented plant material. Preferably, the non-polar fraction according to the present invention does not comprises at least the fibrous material from the at least one fermented plant material

Specific fractions of the one or more fermented plant material may be provided by chromatography. In particular, the specific fraction of isolated fatty acid compounds may be provided by reversed phase chromatography, hydrophobic interaction chromatography, ion exchange chromatography or any combination hereof. Other fractionation processes know to the skilled person may be provided.

In an embodiment of the present invention the composition comprising at least 30% (w/w) non-polar fraction, such as at least 40%, e.g. at least 50%, such as at least 60%, e.g. at least 70%, such as at least 80%, e.g. at least 90%.

In an embodiment of the present invention a fibrous material of the at least one fermented plant material may be removed, or substantially removed, from the composition. In the present context, the term “substantially removed” relates to the presence of less than 5% (w/w) fibrous material, such as less than 3% (w/w) fibrous material, e.g. less than 1% (w/w) fibrous material, such as less than 0.1% (w/w) fibrous material, e.g. about 0% (w/w) fibrous material.

In yet an embodiment of the present invention the composition comprises less than 10% (w/w) fibrous material, such as less than 8% (w/w), e.g. less than 5% (w/w), such as less than 2% (w/w), e.g. less than 1% (w/w).

In an embodiment of the present invention the plant material resulting in the fermented plant material does not include a fruit, a fruit plant and/or derivative thereof.

Preferably, the plant material (resulting in the fermented plant material) may be selected from a proteinaceous plant material. a proteinaceous plant material may be a plant material having a protein content above 5% (w/w), preferably above 10% (w/w), even more preferably above 15% (w/w), even more preferably above 20% (w/w). The protein content of the plant may relative to the ripe version of the plant material.

In an embodiment of the present invention, the one or more plant material(s) resulting in the fermented plant material may be selected from at least one proteinaceous plant material. The plant material, and the proteinaceous plant material, may be selected from at least one of Brassica spp.; seaweed; algae; sun flower; palm; soya, field beans, Lupins; or a combination hereof. Preferably, the fermented plant material, and the proteinaceous plant material, may be selected from Brassica spp.; seaweed/algae; or a combination hereof.

In an embodiment of the present invention, the Brassica spp. may preferably be selected from one or more of rape species; cruciferous vegetables; cabbage species; and/or mustard species. Preferably, the Brassica spp. may preferably be selected from one or more of rape species. Preferably, the rape species is a rapeseed product, such as rapeseed meal, or rapeseed cake, preferably rapeseed cake.

In another embodiment of the present invention, the Brassica spp. may be selected from one or more species such as Brassica napus; Brassica oleracea; Brassica campestris; and/or Brassica rapa.

In a preferred embodiment of the present invention the fermented plant material may be a combination of rape species and seaweed.

In a further embodiment of the present invention, the seaweed and/or algae may be selected from one or more of brown algae, red algae, green algae, such as kelps, Laminaria saccharina (sugar kelp); Laminaria digitata; Laminaria hyperborean; gracilaria; Saccharina latissimi; and Ascophyllum nodosum.

In an even further embodiment of the present invention, the plant material comprises a combination of:
(i) Brassica spp., in particular Brassica napus; or Brassica campestris, and
(ii) seaweed/algae.

In the event the fermented plant material comprises a combination of Brassica spp., in particular Brassica napus; or Brassica campestris; and seaweed/algae, the seaweed/algae may preferably be subjected to the pre-treatment before fermentation to an extent that result in an average diameter which is at most 75% of the average diameter of the Brassica spp., such as at most 50% of the average diameter.

In an embodiment of the present invention, the Brassica spp. may have, optionally after a pre-treatment, an average diameter of 3 mm or less, such as an average diameter of 2 mm or less, such as an average diameter of 1 mm or less, such as an average diameter in the range 25 um to 3 mm, such as 0.1 mm to 2.5 mm, such as an average diameter in the range of 0.5 mm to 2.25 mm, such as an average diameter in the range 1.0 mm to 2 mm. In an embodiment of the present invention, the seaweed/algae may have, optionally after a pre-treatment, an average diameter of 2 mm or less, such as an average diameter of 1.5 mm or less, such as an average diameter of 1 mm or less, such as an average diameter in the range 25 um to 2 mm, such as 0.1 mm to 1.5 mm, such as an average diameter in the range of 0.5 mm to 1.25 mm, such as an average diameter in the range 0.75 mm to 1 mm.

In yet an embodiment of the present invention, the ratio between Brassica spp. and seaweed/algae is at least 1:1, such as at least 1:2, e.g. at least 1:3, such as at least 1:4, e.g. at least 1:5, such as at least 1:6, e.g. at least 1:7, such as at least 1:8, e.g. at least 1:9, such as at least 1:10.

Preferably, the fermented plant material may comprise at least 10% (w/w) rape species and the remaining being seaweed (hence at most 90% (w/w) seaweed; such as at least 20% (w/w) rape species and the remaining being seaweed; e.g. at least 30% (w/w) rape species and the remaining being seaweed; such as at least 40% (w/w) rape species and the remaining being seaweed; e.g. at least 50% (w/w) rape species and the remaining being seaweed; such as at least 60% (w/w) rape species and the remaining being seaweed; e.g. at least 70% (w/w) rape species and the remaining being seaweed; such as at least 80% (w/w) rape species and the remaining being seaweed; e.g. at least 90% (w/w) rape species and the remaining being seaweed.

When the fermented plant material may involve seaweed or algae the method for preparing the fermented composition may be as described herein or as described in WO 2014/206419. This method for preparing fermented seaweed or algae as described in WO 2014/206419 is hereby incorporated by reference.

The one or more plant materials may be subjected to a pre-treatment. Such pre-treatment may involve washing, drying, grinding, cutting, chopping, slicing, and/or fractionizing the plant material before fermentation.

In a preferred embodiment of the present invention the at least one plant material may be subjected to a fermentation process providing at least one fermented plant material, preferably, the at least one fermented plant material may have been subjected to a lactic acid fermentation.

In an embodiment of the present invention the composition according the present invention may be provided by a fermentation process for a fermented plant material (in particular a plant material not being seaweed or algae), the method may comprise the steps of:
(a) providing an inoculums comprising essentially lactic acid bacteria and preferably, the concentration of lactic acid bacteria in the inoculum of step (a) are sufficient to outgrow any bacteria, yeast or moulds present in the product of step (b) and/or step (c);
(b) providing at least one plant material to be fermented;
(c) optionally providing a source of phytase, e.g. in the form of plant material obtained from a crop selected from the group consisting of wheat, rye, trikale, barley, spring barley or a combination thereof;
(d) combining the materials of steps (a), (b) and (c) and fermenting the product of step (b) using the inoculums of step (a) under anaerobic condition at a temperature in the range of 15-48 C, preferably, in the range of 30 to 40 °C., for a period in the range of 24 hours and 14 days, preferably between 2 -12 days, e.g. in the range of 4-11, such as in the range of 5-10 days, e.g. about 7 days.

In an embodiment of the present invention the plant material is a seaweed or an algae, and an extended fermentation time may be necessary. Preferably, the fermentation time of seaweed or algae may be for 10 days or more, e.g. for 15 days or more, such as for 20 days or more, e.g. for 25 days or more, such as for 30 days or more, e.g. for 35 days or more, such as for 40 days or more. In an embodiment of the present invention the fermentation time of seaweed or algae may be in a range of 10-50 days, such as in the range of 12-40 days, e.g. in the range of 15-35 days, such as in the range of 18-30 days, e.g. in the range of 20-24 days.

Preferably, the moisture content during the fermentation step d) is in the range 25-85%, such as in the range 27.5% to 50%, preferably 32 to 38% by weight dry matter (wt%).

In an embodiment of the present invention the fermented plant material is suspended and/or dissolved in a non-polar organic solvent, like methanol, ethanol, propanol, or iso-propanol.

In yet an embodiment of the present invention the fermented plant material may be subjected to a separation step removing all or part of the fibrous material of the fermented plant material before the fermented plant material is suspended and/or dissolved in a non-polar organic solvent. The separation step may result in a liquid fraction comprising the non-polar compound and a solid fraction.

In another embodiment of the present invention the fermented plant material may be subjected to a separation step removing all or part of the fibrous material of the fermented plant material after the fermented plant material is suspended and/or dissolved in a non-polar organic solvent. The separation step may result in a liquid fraction comprising the non-polar compound and a solid fraction.

Preferably the fermented plant material may be provided by a lactic acid fermentation. The lactic acid fermentation may preferably involve at least one lactic acid bacteria.

Furthermore, lactic acid bacteria may produce lactic acid and other metabolic products which contribute to the organoleptic, textural, nutritional and pharmacological profile of the composition according to the present invention.

The industrial importance of the lactic acid bacteria may be evidenced by their generally regarded as safe (GRAS) status, due to their ubiquitous appearance in food and their contribution to the healthy microflora of human mucosal surfaces. The genera that comprise the lactic acid bacteria, and which may be used in the present invention, are Lactobacillus, Leuconostoc, Pediococcus, Lactococcus, and Streptococcus, Aerococcus, Carnobacterium, Enterococcus, Oenococcus, Teragenococcus, Vagococcus, and Weisella; these genera belong to the order Lactobacillies.

In the present invention, the one or more lactic acid bacterial strain(s) provided in step (iii) and used for fermentation may be selected from lactic acid bacteria selected from the group consisting of Lactobacillus, Leuconostoc, Pediococcus, Lactococcus, and Streptococcus, Aerococcus, Carnobacterium, Enterococcus, Oenococcus, Teragenococcus, Vagococcus, and Weisella. Preferably, the one or more lactic acid bacterial strain(s) used for fermentation may be selected from lactic acid bacteria selected from the group consisting of lactic acid bacteria of the genus Enterococcus, Lactobacillus, Pediococcus or Lactococcus, or combinations thereof.

In an embodiment of the present invention, the one or more lactic acid bacterial strain(s) used for fermentation may be selected from the group consisting of one or more Enterococcus spp., Lactobacillus spp., Lactococcus spp., Pediococcus spp., and a combination hereof. Preferably, the one or more lactic acid bacterial strain used for fermentation may be selected from the group consisting of one or more one Enterococcus faecium, Lactobacillus rhamnosus, Lactobacillus plantarum, Pediococcus acidililactili, Pediococcus pentosaceus, Lactococcus Lactis, Lactococcus Cremoris, Lactococcus Diacetylactis, Leuconostoc Cremoris and a combination hereof.

In a further embodiment, the one or more lactic acid bacterial strain(s) used for fermentation comprise Lactobacillus plantarum, Enterococcus faecium and/or Lactobacillus rhamnosus.

In still another embodiment, the one or more lactic acid bacterial strain(s) used for fermentation comprise one or more of Enterococcus faecium MCIMB 30122, Lactobacillus rhamnosus NCIMB 30121, Pediococcus pentosaceus HTS (LMG P-22549), Pendiococcus acidilactici NCIMB 30086 and/or Lactobacillus plantarum LSI (NCIMB 30083) or a combination hereof.

In order to increase productivity and effectivity two or more lactic acid bacterial strains may be provided, such as three or more lactic acid bacterial strains, e.g. four or more lactic acid bacterial strains, such as 7 or more lactic acid bacterial strains, e.g. 10 or more lactic acid bacterial strains, such as 15 or more lactic acid bacterial strains, e.g. 20 or more lactic acid bacterial strains, such as 25 or more lactic acid bacterial strains, e.g. 30 or more lactic acid bacterial strains, such as 35 or more lactic acid bacterial strains, e.g. 40 or more lactic acid bacterial strains.

One way to conduct the fermentation process of the at least one plant material may be as described in PCT/EP2016/076952 which method of fermenting at least one plant material is hereby incorporated by reference.

The non-polar compound and/or the one or more fatty acid compounds may be a complex mixture of different fatty acid compound structures, or modified fatty acid compound structures.

In an embodiment of the present invention the non-polar compound, or the fatty acid compounds, may be produced during the lactic acid fermentation. Hence, it is preferred that the fatty acid compounds are not chemically produced and added and/or that the fatty acid compound is not naturally occurring in the plant material.

The fermented composition may be in the form of a liquid or a powder.

In the present context, the term “non-polar compound” relates to a compound dissolvable in a non-polar solvent. Polar solvents dissolve polar compounds best and non-polar solvents dissolve non-polar compounds best. Strongly polar compounds like sugars (e.g. sucrose) or ionic compounds, like inorganic salts (e.g. table salt) dissolve only in very polar solvents, like water; while non-polar compounds like fatty acids, dissolve only in non-polar organic solvents like methanol, ethanol, propanol, or iso-propanol.

The compound according to the present invention has surprisingly shown to have anti-inflammatory activity. The anti-inflammatory activity was demonstrated as a suppression of the IL-12, IL-1β, and/or IL-6 activity; and/or suppression of tumor necrosis factor alpha (TNF-alpha or TNF-α) activity. The suppression in IL-12, IL-1β, and/or IL-6; activity and/or in TNF-alpha activity may be provided by reducing the amount of TNF-alpha or IL-12, IL-1β, and/or IL-6 measured, in TNF-alpha or IL-12, IL-1β, and/or IL-6 activity, or in TNF-alpha or IL-12, IL-1β, and/or IL-6 detected extracellularly and/or intracellular in an assay performed with a suppressor, compared to the assay performed without the suppressor.

Furthermore, the compound according to the present invention has surprisingly shown to have anti-inflammatory activity which may be demonstrated by an upregulation of one or more of interleukin-10, interleukin-1Ra, interleukin-4, interleukin-11, interleukin-13; or transforming growth factor beta (TGF-β). In particular, an upregulation of IL-10. This upregulation may be demonstrated by an upregulation in an increased amount measured, in the activity detected extracellularly and/or intracellular.

Preferably, the compound according to the present invention has surprisingly shown to have an anti-inflammatory activity by:
suppression of interleukin-12, interleukin-1β, and/or interleukin-6 activity; and/or tumor necrosis factor alpha (TNF-alpha or TNF-α) activity; and
upregulation of interleukin-10, interleukin-1Ra, interleukin-4, interleukin-11, interleukin-13; and/or transforming growth factor beta (TGF-β); when administered to a mammal.

A preferred embodiment of the present invention relates to an anti-inflammatory substance comprising the composition according to the present invention, for use in the treatment, alleviation and/or prophylaxis of an inflammatory disease or disorder in a mammal.

In the context of the present invention, the term “treatment” relates to the use of the composition or the non-polar or the fatty acid compound according to the present invention, in an attempt to cure or mitigate a disease, a condition, or an injury in a mammal.

The term “alleviation” used in the present invention relates to the action of the composition or the non-polar or the fatty acid compound according to the present invention, to make a disease, a condition or an injury less intense and/or reduce symptoms in a mammal.

In the context of the present invention the term “prophylaxis” relates to the use of the composition or the non-polar or the fatty acid compound according to the present invention, in an attempt to prevent a disease, a condition or an injury in a mammal and/or for the protective treatment of a mammal.

The composition, the non-polar and/or the fatty acid compound according to the present invention showed to have strong activity against inflammation. In particular, the anti-inflammatory substance according to the present invention may provide a significant suppression of interleukin-12 (IL-12) and/or a significant suppression of TNF-alpha.

In an embodiment of the present invention the IL-12 activity of the infected tissue may be suppressed by at least 30% relative to un-treated tissue and/or un-treated mammal; such as at least 40%; e.g. at least 50%; such as at least 60%; e.g. at least 70%; such as at least 80%; e.g. at least 90%; such as at least 95%; e.g. at least 98%.

In a further embodiment of the present invention the TNF-alpha activity of the infected tissue may be suppressed by at least 30% relative to un-treated tissue and/or un-treated mammal; such as at least 40%; e.g. at least 50%; such as at least 60%; e.g. at least 70%; such as at least 80%; e.g. at least 90%; such as at least 95%; e.g. at least 98%.

IL-12 and TNF-alpha are some of the most important cytokines and considered key players in the regulation of T cell responses. These responses are orchestrated by monocytes, macrophages, and dendritic cells which produce the various cytokines of the IL-12 family and the TNF-alpha family in response to infection or inflammation.

By suppressing the IL-12, interleukin-1β, and/or interleukin-6 and TNF-alpha cytokines the compound and/or the fatty acid compound according to the present invention may have direct effect on the inflammation rather than an indirect effect where high amounts of cytokines, e.g. IL-12, interleukin-1β, and/or interleukin-6 and TNF-alpha, are produced in the infected tissue and the side effects of this indirect effect traditionally observed may be avoided.

Hence, without being bound by theory it is assumed that the compound, the non-polar and/or the fatty acid compound according to the present invention may act in the defence against an inflammation, without the need to activated the monocytes, macrophages, and dendritic cells to produce the various cytokines of the IL-12 family and the TNF-alpha family in response to various inflammations.

The composition, the non-polar and/or the fatty acid compound according to the present invention showed to have strong activity against inflammation. In particular, the anti-inflammatory substance according to the present invention may provide a significant upregulation of interleukin-10 (IL-10).

In an embodiment of the present invention the IL-10 activity of the infected tissue may be upregulated by at least 30% relative to un-treated tissue and/or un-treated mammal; such as at least 40%; e.g. at least 50%; such as at least 60%; e.g. at least 70%; such as at least 80%; e.g. at least 90%; such as at least 95%; e.g. at least 98%.

In an embodiment of the present the inflammatory disease or disorder may be selected from a chronic inflammatory related disease in a mammal.

In a further embodiment of the present invention the inflammatory disease or disorder may be selected from the group consisting of diabetes, like type 2 diabetes; obesity; cardiovascular diseases; rheumatoid arthritis; osteoarthritis; multiple sclerosis; artherosclerosis; scleroderma, e.g. systemic sclerosis; lupus; systemic lupus erythematosus (SLE); (acute) glomerulonephritis; asthma, such as asthma bronchiale; chronic obstructive pulmonary diseases (COPD); respiratory distress-syndrome (ARDS); inflammatory bowel disease (e.g., Crohn's Disease); colitis (e.g. ulcerative colitis); vasculitis; uveitis; dermatitis; atopic dermatitis (e.g., inflammatory dermatitis); rhinitis (allergica); allergic conjunctivitis; myasthenia gravis; sclerodermitis; sarcoidosis; psoriatic arthritis; ankylosing spondylitis; juvenile idiopathic arthritis; Graves disease; bacterial infections; Sjogren's syndrome; and Behget disease.

In an embodiment of the present invention, the mammal is a human or an animal, preferably, the animal is a domestic animal e.g. dog, cat, horse, cow, pig, chicken, sheep, or goat.

It should be noted that embodiments and features described in the context of one of the aspects or of one of the embodiments of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

Compound analysis of the fermented plant material (comprising a Methanol extract (80% methanol) of the combination of fermented rape species (rape seed) and seaweed (Laminaria spp.)) were identified by subjecting a sample to untargeted metabolomics analysis by Ultra High Pressure Liquid Chromatography coupled to a Q-TOF mass spectrometer (UHPLC-Q-ToF-MS).

Chromatography was performed on a Dionex UltiMate® 3000 Quaternary Rapid Separation UHPLC+focused system (Thermo Fisher Scientific, Germering, Germany).

Separation was achieved on a Kinetex 1.7 u XB-C 18 column (100×2.1 mm, 1.7 μm, 100 Å, Phenomenex, Torrance, CA, USA). Formic acid (0.05%) in water and acetonitrile (supplied with 0.05% formic acid) were employed as mobile phases A and B, respectively. Gradient conditions were as follows: 0.0-0.5 min, 2% B; 0.5-14.0 min 2-20% B; 14.0-20.0 min 20-45% B, 20.0-24.5 min 45-100% B, 24.5-26.5 min 100%, 26.5-26.55 min 100-2% B and 26.55-30.0 min 2% B. The mobile phase flow rate was 300 μl min−1. The column temperature was maintained at 25° C. Four wavelengths (205 nm, 220 nm, 250 nm and 390 nm) were monitored by a UV-VIS detector.

The liquid chromatography was coupled to a Compact micrOTOF-Q mass spectrometer (Bruker, Bremen, Germany) equipped with an electrospray ion source (ESI) operated in positive or negative ionization mode. The ion spray voltage was maintained at −3900 V in negative mode. Dry temperature was set to 250° C. and dry gas flow was set to 8 L min−1. Nebulizing gas was set to 2.5 bar and collision energy to 15 eV. Nitrogen was used as dry gas, nebulizing gas and collision gas.

The m/z range was set to 50-1400. AutoMSMS mode was used to obtain MS and MS/MS spectra of the three most abundant ions present at each time point with smart exclusion to also include less abundant ions. All files were calibrated based on compound spectra collected from Na+-formiate clusters at the beginning of each run.

### Results

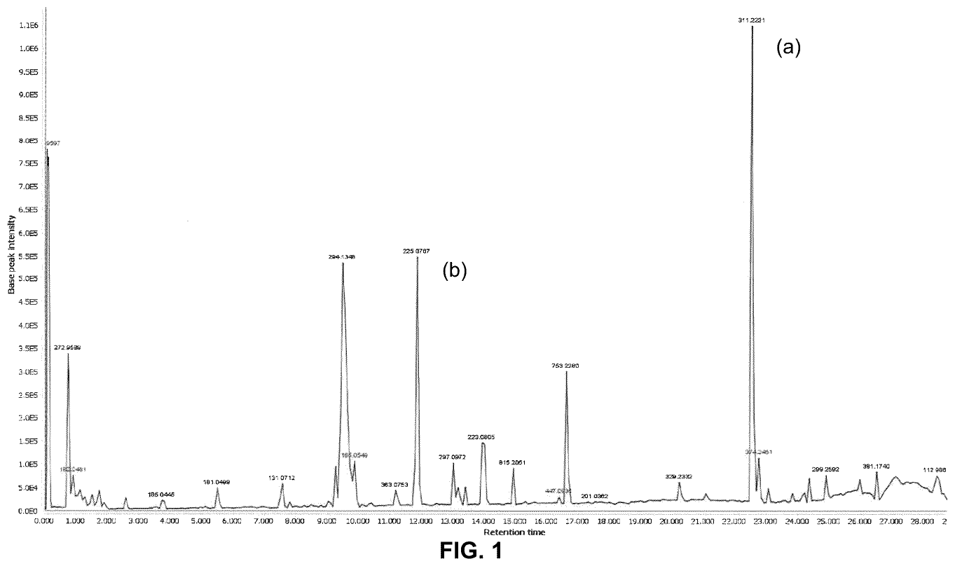

FIG. 1 demonstrates a LC-QToF chromatogram illustrating a metabolite profiling of the fermented plant material and illustrates two significant peak one being most significant.

Analyzing the LC-QTOF chromatogram illustrates that the fermented combination product (rape and seaweed) shows a dominating peak (peak (a) in FIG. 1) that has a “mass to charge ratio” (m/z) of (in negative mode):
MS1 mass=311.2228
Retention time: 22.52 min (under the set conditions)
It is estimated that this compound is a fatty acid, in particular a modified fatty acid.

A second dominating peak (peak (b) in FIG. 1) was found having a “mass to charge ratio” (m/z) of (in negative mode):
MS1 mass=225.0772
Retention time: 11.86 min (under the set conditions)
From comparative studies with pure samples of HDMPPA this second dominating
peak showed to be HDMPPA, 3-(4′-hydroxyl-3′,5′-dimethoxyphenyl)propionic acid (a kimchi compound).

### Example 2

### Preparation of Complex Plant Material Extract

The fermented plant material comprising the combination of rape species (rape seed) and seaweed (Laminaria spp.) were extracted by mixing 100 mg plant material with 1 ml 80% methanol, followed by centrifugation and filtering of the supernatant (0.22 μM). Next, extracts were evaporated in a centrifugal evaporator and pellets were re-solubilized in DMSO to a concentration of 100 mg/ml. These DMSO stocks were diluted and used in cell assays.

### Preparation of a Non-polar Fraction

The non-polar fraction is provided from a fermented plant material comprising the combination of rape species (rape seed) and seaweed (Laminaria spp.).

An 80% methanol extract of the fermented plant material was diluted 3× with water to get a 26.7% methanol extract. This extract was passed over an activated (with 100% methanol) and equilibrated (26.7% methanol) Strata C18 SPE 500 mg column, after which increasing concentrations of methanol was passed over the column to elute compounds with different polarities. The fraction collected after eluting with 90% methanol was evaporated and re-solubilized in DMSO to a concentration of 100 mg/ml. This fraction constitutes the non-polar fraction according to the present invention and used in subsequent cell assays.

### Preparation of Further Comparative Samples

Crambene (CAS No. 6071-81-4, breakdown product of the main glucosinolate in rapeseed: progoitrin), sinapic acid (CAS No. 530-59-6, main free phenolic acid in rapeseed), dihydrosinapic acid (CAS No. 14897-78-0, the “kimchi compound”, very similar to sinapic acid, produced in large amounts during fermentation of rapeseed meal) and “unk comp” (CAS No. 19895-95-5, Kaempferol 3-O-β-D-sophoroside, enriched during fermentation of rapeseed meal) were all commercially available and were included in cell assys as pure compounds solubilized in water (crambene) or DMSO (sinapic acid and dihydrosinapic acid). The comparative samples were used in subsequent cell assays.

Protocol for Stimulation of Bone Marrow-derived Dendritic Cells (bmdcs) With Above Extracts/Samples

Bone marrow from C57BL/6 mice (Tactonic, Lille Skensved, Denmark) was flushed out from the femur and tibia and washed. 3×10⁵ bone marrow cells were seeded into 10 cm Petri dishes in 10 ml RPMI 1640 (Sigma-Aldrich, St. Louis, MO, USA) containing 10% (v/v) heat inactivated fetal calf serum supplemented with penicillin (100 U ml−1), streptomycin (100 mg ml−1), glutamine (4 mM), 50 mm 2-mercaptoethanol (all from Cambrex Bio Whittaker) and 15 ng ml−1 murine GM-CSF (harvested from a GM-CSF transfected Ag8.653 myeloma cell line). The cells were incubated for 8 days at 37° C. in 5% CO 2 humidified atmosphere. On day 3, 10 ml of complete medium containing 15 ng ml−1 GM-CSF was added. On day 6, 10 ml were removed and replaced by fresh medium. Non-adherent, immature DC were harvested on day 8. Afterwards, immature DCs (2×10⁶ cells ml−1) were resuspended in fresh medium, and 500 μl well-1 were seeded in 48-well tissue culture plates (Nunc, Roskilde, Denmark).

The day of the stimulation, the extracts/samples have been added to BMDCs with a volume of 100 μl for each well of the 48-well plates, and to a final concentration of 100 μg/ml, and the cells have been incubated for 1 h at 37° C. with 5% CO₂. Two controls have been included: one just with RPMI media, and one with the corresponding amount of DMSO present in 100 μg/ml of the extracts (in any case not exceeding the 0.1 % v/v). After 1 h of incubation with cells and extracts/samples, two different kind of stimulus have been added to cells: 1 μg/ml of LPS from Escherichia coli 0127 :B8 (Sigma-Aldrich), or L. acidophilus NCFM at a multiplicity of infection (MOI) of 1. Controls of LPS and NCFM alone and together with DMSO have been included. The cells have been then incubated for 20 h. After that the supernatant of each condition and combination, tested in triplicates, has been collected and stored at −80° C. until the day of the ELISA assay. The protein production of IL-12 and TNF-α was analysed using commercially available ELISA kits (Biotechne, UK).

### Results

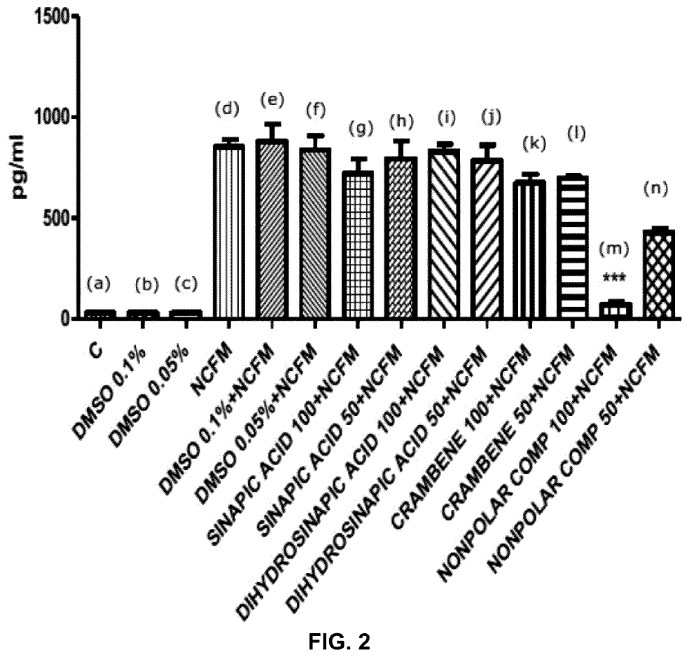

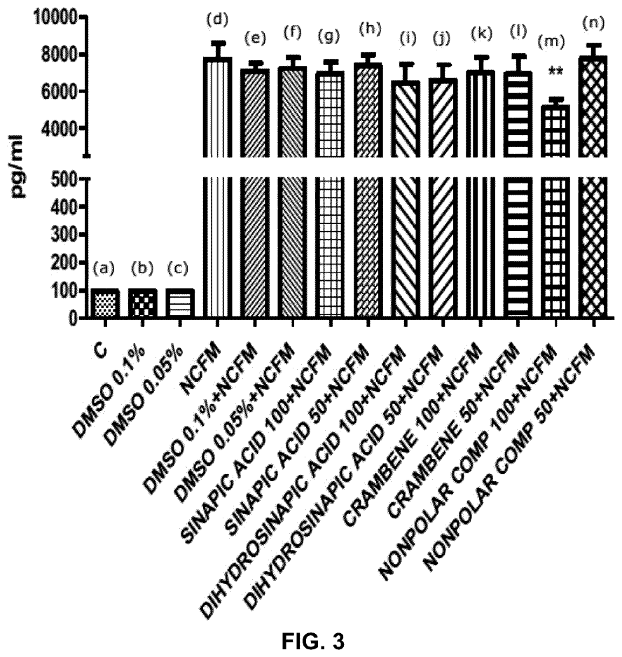

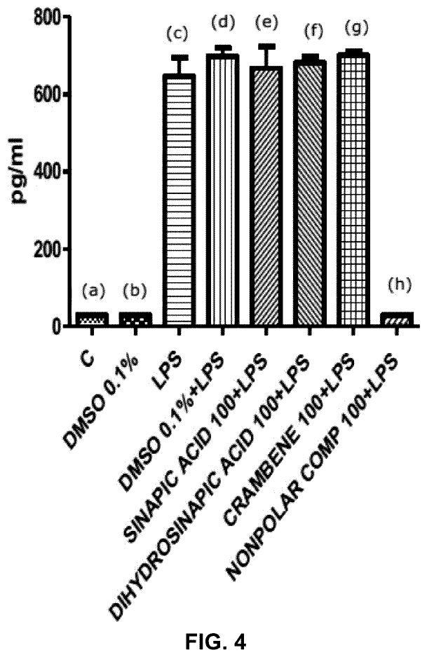

The results of the subsequent cell assays show a significant anti-inflammatory effect of the fermented plant material where inflammation was induced by NCFM MOI (see FIGS. 1 and 2) or by LPS (see FIGS. 3 and 4).

FIG. 2 shows the effect of the non-polar fraction obtained from the fermented plant material comprising the combination of rape species (rape seed) and seaweed (Laminaria spp.) on inflammation induced by NCFM MOI. The figure shows that the non-polar fraction according to the present invention has a strong suppression on IL-12 activity and expression (being suppressed to less than 10% of the IL-12 activity found in the other comparative samples), see column (m) and column (n), comprising the non-polar fraction relative to in particular column (d), which is the control showing the effect of the untreated infected sample, column (d). Column (m) and column (n) also demonstrates that a dose response effect in the suppression of IL-12 is to be found with the non-polar fraction according to the present invention. Column (m) has twice the amount of non-polar fraction than the amount found in the test illustrated in column (n) and the effect of the higher amount of non-polar fraction is about 5 times higher.

FIG. 3 shows the effect of the non-polar fraction according to the present invention on inflammation induced by NCFM MOI. The figure shows that the non-polar fraction according to the present invention has a strong suppression on the TNF-alpha activity and expression see column (m) relative to column (d) which is the control showing the effect of the untreated infected sample, column (d).

FIG. 4 shows the effect of the non-polar fraction according to the present invention on inflammation induced by LPS (Lipopolysaccharides). The figure shows that the non-polar fraction according to the present invention has a strong suppression on the IL-12 activity and expression see column (h) relative to column (c) which is the control showing the effect of the untreated infected sample, column (c).

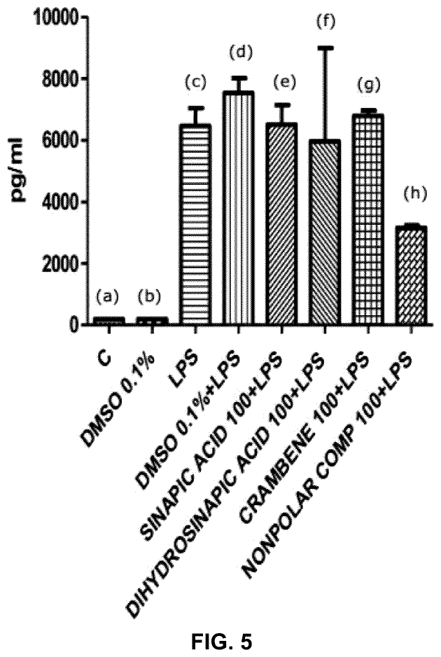

FIG. 5 shows the effect of the non-polar fraction according to the present invention on inflammation induced by LPS (Lipopolysaccharides). The figure shows that the non-polar fraction according to the present invention has a strong suppression on the TNF-alpha activity and expression see column (h) relative to column (c) which is the control showing the effect of the untreated infected sample, column (c).

### REFERENCES

WO 2014/206419
PCT/EP2016/076952

1-15. (canceled)

16. A method for suppressing one or more of interleukin-12, in a mammal, by administering a composition to the mammal comprising a non-polar fraction obtained from at least one fermented plant material, wherein the at least one plant material is selected from at least one proteinaceous plant material, wherein the proteinaceous plant material is selected from at least one of Brassica spp., seaweed, algae, sunflower, palm, soya, field beans, Lupins, or a combination thereof, and wherein the at least one plant material has been subjected to a lactic acid fermentation for a period of 1-14 days, or for seaweed or algae for an extended period of an additional 10-15 days.

17. The method according to claim 16, wherein the non-polar fraction comprises one or more fatty acid compounds.

18. The method according to claim 16, wherein the non-polar fraction comprises one or more modified fatty acid compounds.

19. The method according to claim 16, wherein the non-polar fraction comprises one or more fatty acid compounds or modified fatty acid compounds selected from a C18-fatty acid compound, a modified C18-fatty acid compound, a linolenic acid compound, or a modified linolenic acid compound.

20. The method according to claim 16, wherein the composition is an isolated composition separated from the at least one plant material.

21. The method according to claim 16, wherein the lactic acid fermentation involves at least one lactic acid bacteria.

22. The method according to claim 16, wherein a fibrous material of the at least one plant material has been removed, or substantially removed, from the composition.

23. The method according to claim 16, wherein the plant material is a combination of rape species and seaweed.

24. The method according to claim 23, wherein the plant material comprises at least 10% (w/w) rape species and at most 90% seaweed.
